(19) United States
(12) Patent Application Publication
(10) Pub. No.: US 2009/0187195 A1
(43) Pub. Date: Jul. 23, 2009

(54) FOREIGN OBJECT EXTRACTOR

(76) Inventors: Joseph Berk, Owings Mill, MD (US); Jon L. Roberts, Great Falls, VA (US)

Correspondence Address: The Marbury Law Group, PLLC, 11800 SUNRISE VALLEY DRIVE, SUITE 1000, RESTON, VA, 20191, US

(21) Appl. No.: 12/017,148

(22) Filed: Jan. 21, 2008

(51) Int. Cl. A61B 17/22

(52) U.S. Cl. 606/127

(57) ABSTRACT

The foreign object extractor has an elongated hollow tube with a central lumen through its entire length providing fluid connection between an engaging end and a vacuum source. The engaging end has a nominally round transverse cross-section and is shaped to engage a foreign object. The engaging end flares so as to expose the internal surface of the elongated hollow tube for engagement with the foreign object. An adhesive is disposed on the exposed internal flared surface of the distal end. Pneumatic suction is applied through the lumen of the extractor and the suction serves to bring the foreign object into contact with the adhesive coated surface of the extractor to form a bond between the extractor and the object to be extracted.

### FIELD OF THE INVENTION

The present invention is directed to the art of medical instruments, and more particularly to instruments for removal of objects from within a living body.

### BACKGROUND INFORMATION

Commonly objects become lodged in a living body and need to be removed. Various types and sizes of forceps have been used in the past to seize and extract such embedded objects.

The use of forceps for object extraction can be traumatic to cartilage and soft tissue. Forceps are formed of metal or rigid plastic, which are hard and can cause pain and tearing of tissue even when wielded with the gentlest technique possible.

Thus, what is needed is an extractor instrument that is more pliable than metal or rigid plastic and grips objects securely for extraction.

An endoscopic surgical instrument has been proposed, which relies entirely upon vacuum pressure to maintain a grip on the foreign body. For additional details, refer to U.S. Pat. No. 5,196,003 issued to Bilweis. Because embedded foreign bodies can have irregular shapes or surface textures, vacuum pressure alone is sometimes unreliable in maintaining a grip on the object during extraction. If the object has a hole, such as a bead, the vacuum pressure will be unable to grip the object. Also, because some embedded foreign bodies are easily frangible and because control of the amount of vacuum pressure applied is difficult, reliance on vacuum pressure alone for retention of the object during extraction risks fragmenting the object.

Thus what is needed is an extractor instrument that can secure grip on an object without relying solely on vacuum pressure.

A foreign body extractor has been proposed that has an expandable distal end that sweeps the foreign body out of a passage. For additional details, refer to U.S. Pat. No. 5,454,817 issued to Katz. This extractor is of limited utility since it can only remove a foreign body that is situated with sufficient free space around it to permit the extractor to slide past it before expanding its distal end. Clearly, any foreign body that is wedged tight cannot be addressed with this extractor.

Thus what is needed is an extractor instrument that can apply extracting force to a foreign object without need to slide past the foreign object.

### SUMMARY OF THE INVENTION

The foreign object extractor is an elongated hollow tube with a central lumen through its entire length. A distal engaging end is shaped to engage a foreign object and has a nominally round transverse cross-section. In an embodiment, the shape of the engaging end flares so as to allow a wider cross section and surface area of the end of the tube to contact the foreign object for engagement with the foreign object. An adhesive is disposed on the exposed flared surface of the distal end. Pneumatic suction is applied through the lumen of the extractor and the suction serves to bring the foreign object into contact with the adhesive coated surface of the extractor.

One aspect of the foreign object extractor is that it is more pliable than metal.

Another aspect of the foreign object extractor is the ability to securely grip an object without relying solely on vacuum pressure.

Yet another aspect of the foreign object extractor is the ability to adhesively grip frangible or hollow articles unsuited to vacuum pressure.

Still another aspect of the foreign object extractor is that it applies extracting force to a foreign object without the need to slide past the foreign object.

### BRIEF DESCRIPTION OF THE DRAWINGS

FIG. 1 illustrates a perspective view of a foreign object extractor according to a first embodiment.

FIG. 2 illustrates a perspective view of a foreign object extractor according to a second embodiment.

FIG. 3 illustrates a perspective view of a foreign object extractor according to a third embodiment.

FIG. 4 illustrates a cross section view of a flared engaging end of a foreign object extractor having a moderate flare.

FIG. 5 illustrates a cross section view of a flared engaging end of a foreign object extractor having a narrow flare.

FIG. 6 illustrates a cross section view of a flared engaging end of a foreign object extractor having a wide flare.

FIG. 7 illustrates a detail view of the flare end of the foreign object extractor.

FIG. 8 illustrates a detail view of the flare end of the foreign object extractor having a segmented configuration.

FIG. 8A illustrates a scored foreign body extractor end.

FIG. 9 illustrates a detail view of the flare end of an embodiment of the foreign object extractor that incorporates a fixed angle bend.

FIG. 10 illustrates a detail view of the engaging end of an embodiment of the foreign object extractor that incorporates a manually-bendable member and a pair of paddle-shaped adhesive grippers.

### DETAILED DESCRIPTION

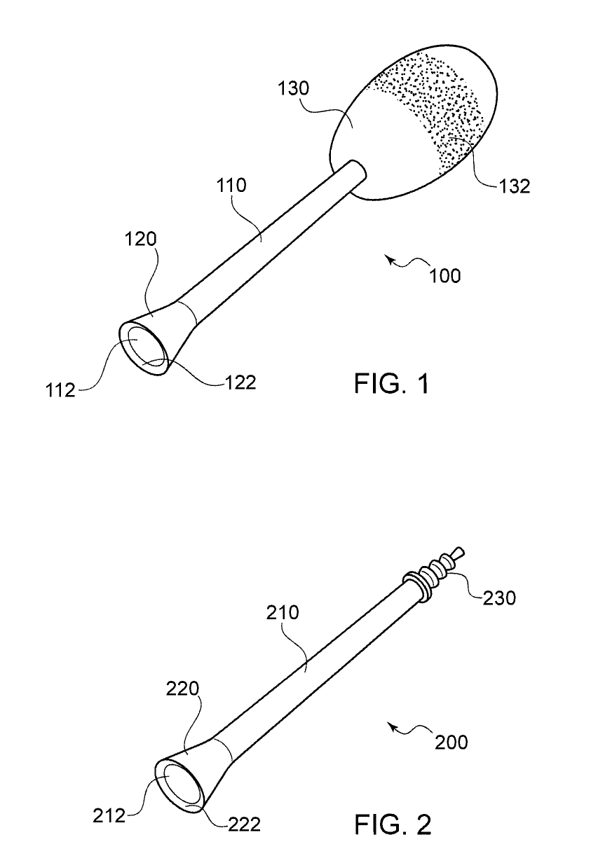

Referring to FIG. 1, a perspective view of a foreign object extractor according to a first embodiment is illustrated. The extractor 100 has an elongate body 110 with a flared engaging end 120. The body 110 is hollow with a lumen 112 extending the entire length of the body 110 from the engaging end 120 to a vacuum bulb 130. The tubular body 110 is resiliently flexible. The rim of the engaging end 120 has an annular sticky surface 122. In this illustrated embodiment the periphery of the vacuum bulb 130 has a textured surface 132 to enhance grip although this is not meant as a limitation.

Although the amount of suction that can be provided at the engaging end 120 is limited by the size of the vacuum bulb 130, this first embodiment extractor 100 has the advantage of being portable so that it is useful away from a sustained vacuum source. Further it is anticipated that this, and the other embodiments illustrated herein are disposable and will be single use only so as to avoid any contamination and transmission of disease or pathogens from one patient to the next.

It should also be noted that in this and other illustrated embodiments, a flared end is illustrated. This is not meant as a limitation since a flared end may not be required for all situations. Yet another embodiment may simply have a tube whose end diameter is the same as the entire tube, but having a sticky end surface that comes in contact with the foreign object.

In addition, it is anticipated that flared ends of varying sizes will be represented by alternate embodiments. The flare size would be selected by the practitioner based on the canal or passage in which the foreign object is embedded and by the shape of the embedded foreign body.

Referring to FIG. 2, a perspective view of a foreign object extractor according to a second embodiment is illustrated. The extractor 200 has an elongate body 210 with a flared engaging end 220. The body 210 is hollow with a lumen 212 extending the entire length of the body 210 from the engaging end 220 to a vacuum fitting 230. The tubular body 210 is resiliently flexible. The inside rim of the engaging end 220 has an annular sticky surface 222.

The vacuum fitting 230 provides for easy connection to a vacuum line of a vacuum pump. This second embodiment extractor 200 provides for application of a continuous vacuum which may be available from a portable vacuum pump or from a vacuum source in a permanent facility such as an operating or emergency room. Such room suction vacuum lines are common infrastructure that may be utilized in a hospital, clinic, urgent care, or trauma center setting.

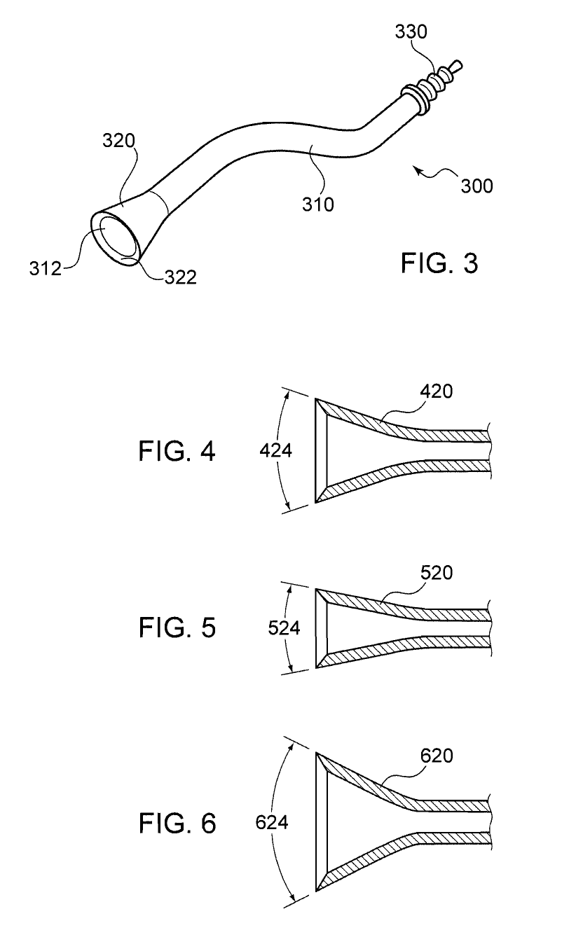

Referring to FIG. 3, a perspective view of a foreign object extractor according to a third embodiment is illustrated. The extractor 300 has an elongate body 310 with a flared engaging end 320. The body 310 is hollow with a lumen 312 extending the entire length of the body 310 from the engaging end 320 to a vacuum fitting 330. The tubular body 310 is plastically flexible. The rim of the engaging end 320 has an annular sticky surface 322.

Similarly as in the second embodiment, the vacuum fitting 330 provides for easy connection to a vacuum line of a vacuum pump or a permanent vacuum source.

Referring to FIG. 4, a cross section view of a flared engaging end 420 of a foreign object extractor having a moderate flare is illustrated. The flare angle 424 is selected to provide positive engagement of the engaging end 420 with medium sized objects such as those the size of marbles. The illustrated moderate flare angle 424 is approximately 45 degrees, but a moderate flare angle may range from 37 to 53 degrees.

Referring to FIG. 5, a cross section view of a flared engaging end of a foreign object extractor having a narrow flare is illustrated. The flare angle 524 is selected to provide positive engagement of the engaging end 520 with small sized objects such as those the size of tic-tac® mints. The illustrated narrow flare angle 524 is approximately 30 degrees, but a narrow flare angle may range from 20 to 37 degrees with other flare angles possible as well, again depending on the circumstances.

Referring to FIG. 6, a cross section view of a flared engaging end of a foreign object extractor having a wide flare is illustrated. The flare angle 624 is selected to provide positive engagement of the engaging end 620 with larger sized objects such as those the size of batteries. The illustrated wide flare angle 624 is approximately 60 degrees, but a wide flare angle may range from 53 to 75 degrees.

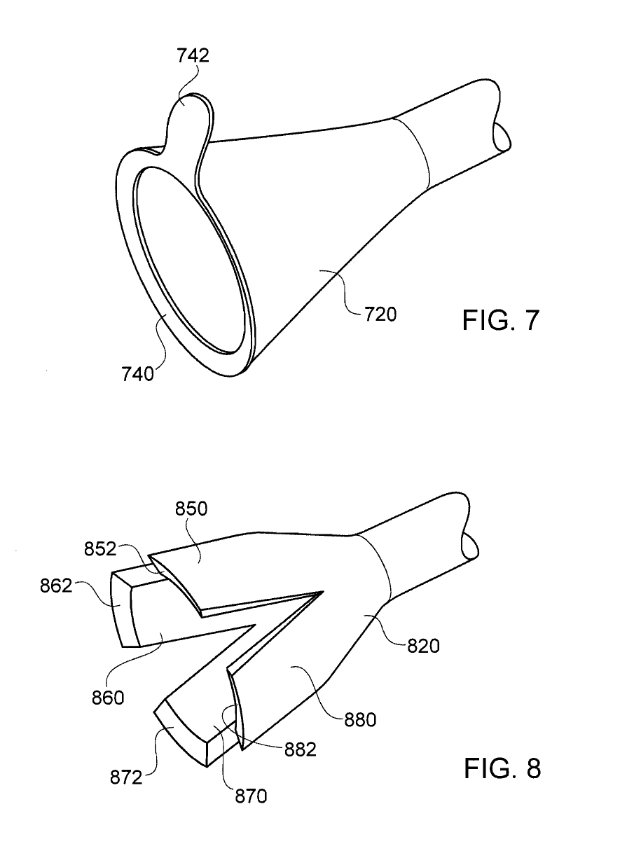

Referring to FIG. 7, a detail view of the flared engaging end 720 of the foreign object extractor is illustrated. A protective cover 740 is disposed over the inner rim of the engaging end 720 to cover up and protect the sticky surface (not shown in this view; refer to FIGS. 1-3) prior to use. The protective cover 740 has a tab 742 sized to be grasped so that the protective cover 740 can be pulled off to expose the sticky surface immediately prior to use of the extractor.

In the embodiments illustrated in FIGS. 1-7 and 9, various flare angles are illustrated. This is not meant as a limitation however since the end of the flare or non-flared tube may simply be perpendicular to the axis of the tube and can be equally useful without a flare or flare angle of the end.

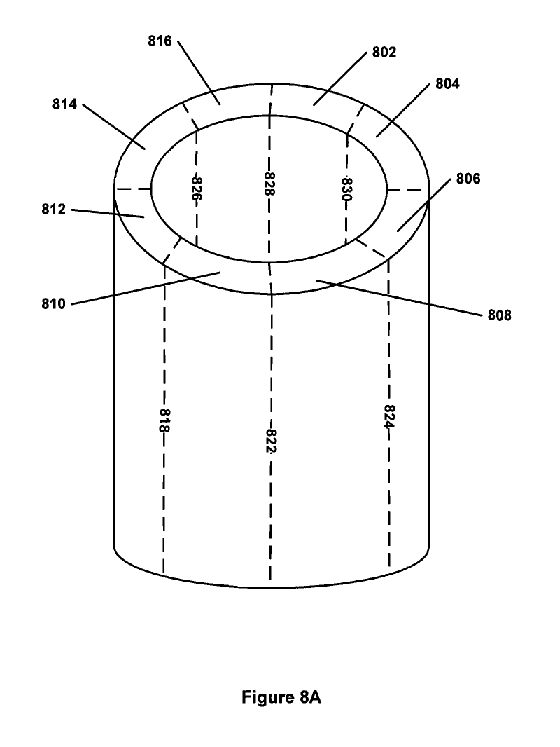

Referring to FIG. 8, a detail view of the engaging end of the foreign object extractor having a segmented configuration is illustrated. The flared engaging end 820 is divided at its extreme periphery into four distinct engaging segments 850, 860, 870, 880 that are resiliently flexible so as to move independently of one another to a degree. The extreme end of each of the engaging segments 850, 860, 870, 880 has a respective sticky surface 852, 862, 872, 882.

While the segmentation of the engaging end 820 will affect the strength of the vacuum force applied to the foreign object, the vacuum force will nevertheless provide some degree of force to cause engagement of a foreign body with the sticky surfaces 852, 862, 872, 882 on the ends of the engaging segments 850, 860, 870, 880. The advantage of segmentation of the engaging end 820 is that it increases the likelihood of obtaining a secure engagement with a foreign object that has an unforeseen, irregular shape. In addition, this segmented shape may be used to further engage surfaces of a more regular object (i.e., such as the surface of a marble, etc.) as illustrated above. It will be appreciated by those skilled in the art that the number of engaging segments illustrated is not meant as a limitation. Depending on the specific situation, the age of the patient, the object to be extracted and other factors, the number of segments of the segmented end will vary.

The segmented end illustrated in FIG. 8 may also comprise sticky surfaces on the interior portion of the engaging segments 850, 860, 870, 880. Further, this segmented end may surround a tube which itself has a sticky surface on its end similar to that illustrated in FIG. 2, 222 in order to have further sticky surfaces to engage the foreign object to be extracted.

Referring now to FIG. 8A, yet another embodiment of the foreign object extractor is illustrated. As illustrate in FIG. 8A, the segmented end can initially be scored into a variable number of engagement segments 802, 804, 806, 808, 810, 812, 814, and 816. The number of segments illustrated is not meant as a limitation. The scored end can then be inserted into an orifice from which a foreign object is to be extracted. The scored end will then separate into segments along score lines 818, 822, 824, 826, 828, and 830 (note several score lines are not visible in this illustration) to engage the foreign body to be extracted. In this manner, segments will not become bent or otherwise pulled apart as they are being inserted and before they engage the foreign body. This embodiment as illustrated also comprises various end and internal sticky surfaces, as described herein, to allow attachment to and extraction of foreign bodies.

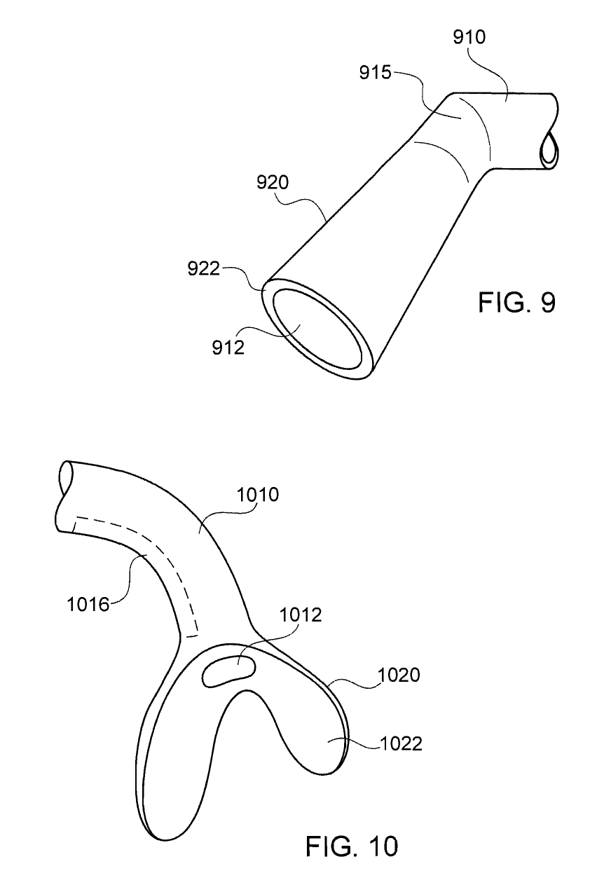

Referring to FIG. 9, a detail view of the engaging end of a fourth embodiment of the foreign object extractor that incorporates a fixed angle bend 915 is illustrated. The flared engaging end 920 is connected to the elongate body 910 with a fixed angle bend 915 so as to allow lumen 912 and annular sticky surface 922 to reach around bends or corners. While illustrated as an approximate 45 degree angle, this is not meant as a limitation and the fixed angle bend can be manufactured to any suitable or desired angle between 10 and 90 degrees. The fixed angle bend 915 has utility with elongate bodies 910 that are rigid, resiliently (i.e., elastically) flexible or deformable, or plastically flexible or deformable.

Referring to FIG. 10, a detail view of the engaging end of a fifth embodiment of the foreign object extractor that incorporates a manually-bendable member 1016 and a pair of paddle-shaped grippers 1020 is illustrated. The engaging end with paddle-shaped grippers 1020 is connected to the elongate body 1010, which includes a manually-bendable member 1016 adjacent to the gripper end so as to allow lumen 1012 and paddle-shaped sticky surface 1022 to reach around bends or corners. The bendable member can be manually manipulated to any desired angle. The manually-bendable member 1016 has utility with elongate bodies 1010 that are rigid, resiliently (i.e., elastically) flexible or deformable, or plastically flexible or deformable (albeit not to such a degree as the manually-bendable member 1016). Furthermore, the manually-bendable member 1016 can alternately be incorporated on one or both of the paddle-shaped grippers 1020 in order to customize the shape of the gripping end to accommodate the foreign object and/or the body cavity.

While the manually-bendable member 1016 is illustrated as a thin strip of embedded metal, this is not meant as a limitation and the manually-bendable member can take other forms, including but not limited to embedded metal wires, adhesively-attached thin metal strips, adhesively-attached metal wires, and remotely-operated devices (e.g., cable actuators). Furthermore, although disclosed in combination, the manually-bendable member 1016 and paddle-shaped grippers 1020 need not be used in combination and can be used individually in other embodiments.

The transverse cross section of the engaging end of the extractor is nominally circular, although it may be embodied as oval or segmented (refer to the third and fifth embodiments described above). The extractor is made in various sizes to provide choice to the health care practitioner for addressing embedded foreign objects of various sizes and shapes. The size variations are provided by different flare angles for the engaging end (refer to FIGS. 4-6), and is also provided by a straightforward scaling up or down of the overall dimensions (i.e., different diameters).

The engaging end has an adhesive disposed on its outer rim or inner rim surfaces of the flared ends to form the sticky surface. The adhesive is a quick curing type that is kept covered until time of use, at which time the protective cover is removed from the sticky surface and the adhesive begins its curing process. The engaging end of the extractor is brought into contact with the object to be removed and suction is applied through the central lumen of the extractor to forcefully urge the object into intimate contact with the sticky surface. This contact is maintained by continued application of vacuum pressure through the extractor until the adhesive has cured. Suction provides pressure for secure adhesion to the foreign object. The object can then be removed from the living body by pulling on the extractor to which the foreign body has become adhered. Additionally to the adhesion, continued application of suction pressure through the extractor may be used to supplement connection of the extractor to the object.

To handle differing extraction situations, the extractor body may be semi-rigid and bendable or totally flexible. The body may be elastically flexible or plastically flexible. In an alternate embodiment, the extractor may be embodied having a rigid body—the rigidity being advantageous in some extraction situations.

While the foreign object extractor has been described in terms of use with a living body, it will be apparent to those skilled in the art that this same configuration can be used to extract foreign objects from inanimate objects as well, e.g., when small objects fall in cracks or other inaccessible location. Thus the disclosure herein should not be deemed to be limited to removal of objects from living bodies only.

A foreign object extractor for seizing hold of and extracting a foreign object from a living body has been described. It will be understood by those skilled in the art that the present invention may be embodied in other specific forms without departing from the scope of the invention disclosed and that the examples and embodiments described herein are in all respects illustrative and not restrictive. Those skilled in the art of the present invention will recognize that other embodiments using the concepts described herein are also possible. Further, any reference to claim elements in the singular, for example, using the articles “a,” “an,” or “the” is not to be construed as limiting the element to the singular.

What is claimed is:

1. An extractor for seizing and removing a foreign object, the extractor comprising:
a vacuum bulb; and
an elongate body having a lumen extending through the entire length of the body, a first end of the body being an engaging end and a second end of the body opposite the first end being connected to the vacuum bulb so that the vacuum bulb is in fluid communication with the lumen;
wherein the engaging end has an annular sticky surface.

2. The extractor of claim 1, wherein the elongate body is resiliently flexible.

3. The extractor of claim 1, wherein at least a portion of the elongate body is plastically deformable.

4. The extractor of claim 1, wherein the elongate body is substantially rigid.

5. The extractor of claim 1, wherein the engaging end is flared.

6. The extractor of claim 1, wherein the vacuum bulb comprises a textured surface.

7. The extractor of claim 1, wherein the sticky surface comprises adhesive.

8. The extractor of claim 1, wherein the sticky surface comprises a quick curing adhesive, and further comprising a removable cover disposed over the quick curing adhesive, the cover being removable prior to the extractor being used for seizing and removing the foreign object.

9. The extractor of claim 5, wherein the flared end comprises split segments or paddle-shaped grippers.

10. The extractor of claim 1, wherein the engaging end has a substantially circular transverse cross section.

11. An extractor for seizing and removing a foreign object in conjunction with a vacuum line, the extractor comprising:
a vacuum fitting adapted for connection to the vacuum line;
an elongate body having a lumen extending through the entire length of the body, a first end of the body being an engaging end and a second end of the body opposite the first end being connected to the vacuum fitting so that the vacuum fitting is in fluid communication with the lumen;
wherein the engaging end has an annular sticky surface.

12. The extractor of claim 11, wherein the elongate body is resiliently flexible.

13. The extractor of claim 11, wherein at least a portion of the elongate body is plastically deformable.

14. The extractor of claim 11, wherein the elongate body is substantially rigid.

15. The extractor of claim 11, wherein the engaging end is flared.

16. The extractor of claim 11, wherein the sticky surface comprises adhesive.

17. The extractor of claim 11, wherein the sticky surface comprises a quick curing adhesive, and further comprising a removable cover disposed over the quick curing adhesive, the cover being removable prior to the extractor being used for seizing and removing the foreign object.

18. The extractor of claim 15, wherein the flared end comprises split segments or paddle-shaped grippers.

19. The extractor of claim 11, wherein the engaging end has a substantially circular transverse cross section.

20. An extractor for seizing and removing a foreign object in conjunction with a vacuum line, the extractor comprising:
a vacuum fitting adapted for connection to the vacuum line;
a resiliently flexible elongate body having a lumen extending through the entire length of the body, a first end of the body being a flared engaging end and a second end of the body opposite the first end being connected to the vacuum fitting so that the vacuum fitting is in fluid communication with the lumen;
wherein the engaging end is divided into plural resiliently flexible engaging segments, each of the engaging segments having a respective sticky surface.

21. The extractor of claim 20, wherein the sticky surface comprises adhesive.

22. The extractor of claim 20, wherein the sticky surface comprises a quick curing adhesive, and further comprising a removable cover disposed over the quick curing adhesive, the cover being removable prior to the extractor being used for seizing and removing the foreign object.

23. The extractor of claim 20, wherein the engaging segments are movable at least partially independently of one another.

24. The extractor of claim 20, wherein the elongate body includes a bend or bendable element adjacent to the engaging end.

25. The extractor of claim 20, wherein the engaging end is scored prior to use thereby allowing the plural resiliently flexible engaging segments to separate from one another when in contact with the foreign object.

26. The extractor of claim 9, wherein the engaging end is scored prior to use thereby allowing the split segments to separate from one another when in contact with the foreign object.

27. The extractor of claim 18, wherein the engaging end is scored prior to use thereby allowing the split segments to separate from one another when in contact with the foreign object.
